SEQUENCE LISTING (2 sequences)

SEQ ID NO: 1 - protein, 192 aa, synthetic construct
YQVRNSSGLYHVTNDCPNSSIVYEAADAILHTPGCVPCVREGNASRCWVAVTPTVGATRDKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDA

SEQ ID NO: 2 - protein, 363 aa, synthetic construct
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFGKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

(19) United States
(12) Patent Application Publication
(10) Pub. No.: US 2025/0264467 A1
(43) Pub. Date: Aug. 21, 2025

(54) NOVEL HCV NUCLEIC ACIDS AND ENCODED GLYCOPROTEIN ANTIGENS AND METHODS

(71) Applicants: Purdue Research Foundation, West Lafayette, IN (US); The Board Of Trustees Of The Leland Stanford Junior University, Stanford, CA (US)

(72) Inventors: Devika Sirohi, West Lafayette, IN (US); Zhen-Yong Keck, Redwood City, CA (US); Steven K.H. Foung, Stanford, CA (US); Richard J. Kuhn, West Lafayette, IN (US)

(21) Appl. No.: 19/053,963

(22) Filed: Feb. 14, 2025

Related U.S. Application Data
(60) Provisional application No. 63/555,320, filed on Feb. 19, 2024.

(51) Int. Cl. G01N 33/569; C07K 14/005; C12N 15/85; G01N 33/50

(52) U.S. Cl. CPC G01N 33/56983; C07K 14/005; C12N 15/85; G01N 33/5023; G01N 33/5032; C12N 2770/24222; G01N 2333/186

(57) ABSTRACT

The present disclosure provides for novel nucleic acid constructs and methods for expressing HCV E2 protein on a cell surface as an active antigen. The present disclosure provides for a method for evaluation of protein expression. Vaccine formulations, constructs and expression vectors are all within the scope of this disclosure.

### CROSS-REFERENCE TO RELATED APPLICATION

This Application relates to and claims the benefit of U.S. provisional application 63/555,320, filed on Feb. 19, 2024. The contents of which are expressly incorporated herein by reference in its entirety into this present disclosure.

### STATEMENT OF GOVERNMENT SUPPORT

This invention was made with government support under AI159840 awarded by the National Institutes of Health. The government has certain rights in the invention.

### STATEMENT OF SEQUENCE LISTING

A computer-readable form (CRF) document of the Sequence Listing is submitted with this application. The document, entitled 70514.xml (4 kb), was generated on Feb. 6, 2025. Applicant states that the content of the computer-readable form is the same and the information recorded in computer readable form is identical to the written sequence listing.

### TECHNICAL FIELD

The present application relates to compositions of matter and methods for expressing novel Hepatitis C virus (HCV) proteins, and methods for assay and evaluation of protein expression. In particular the present application relates to cell surface expressible HCV E2 glycoproteins designed to induce a robust immune response against the pathogen and methods for the evaluation of protein expression as compared with cell surface expression or secretion.

### BACKGROUND

This section introduces aspects that may help facilitate a better understanding of the disclosure. Accordingly, these statements are to be read in this light and are not to be understood as admissions about what is or is not prior art.

Despite the availability of >95% effective direct-acting antiviral medications against HCV (Hepatitis C virus), a vaccine is urgently needed. Prophylaxis via vaccination, point-of-care diagnostics, and accessibility to treatment are needed in tandem to successfully tackling this virus, which according to WHO causes 1.5 million new infections each year. More than 70% of the cases do not resolve acute HCV infection, instead progress to an indefinite chronic phase which over years, often decades, can result in liver disease, cirrhosis, hepatocellular carcinoma, and death. 58 million people are estimated per WHO to be suffering from chronic infection worldwide, ˜2.4 million of these reside in USA (CDC), with a large fraction unaware of their diagnosis. HCV claimed ˜290,000 lives globally in 2019 according to WHO estimates and is the leading cause for liver transplantation in USA. The opioid crisis has led to a dramatic surge in infections and reinfections in North America, and thus there is a pressing humanitarian need and market opportunity for a vaccine against this blood-borne pathogen in both developing and developed countries.

Many barriers have impeded the development of a successful vaccine against HCV thus far, including the vast genetic diversity of the virus (8 genotypes with 30-35% variability in nucleotide sequence), robust immune evasion tactics employed by the virus (such as glycan cloud masking epitopes and hypervariable antigenic regions), emergence of escape variants under immunological pressure and technological obstacles including sub-optimal HCV cell culture system and inadequate animal models. There has also been a lack of clarity on the correlates of protection; the field has pivoted in recent years from a T cell exclusive narrative to the point of view that an early and broad antibody response is also required for effective resolution of the virus.

Vaccine antigen optimization for invoking a potent and wide B cell antibody response is not straightforward. The conventional approach using an inactivated vaccine platform is not readily viable due to low titers of virus achieved in cell culture, virions association with host lipoproteins/lipids and the heterogeneity of these lipovirus particles. HCV E2 surface glycoprotein is a major target of neutralizing antibodies; E1 protein is important for reciprocal folding of E2 and anchoring it to the membrane, essential for stabilizing critical epitope(s) on E2 and itself is a target, albeit minor, for neutralizing antibodies. The development of subunit vaccines comprising the full-length E1 and E2 glycoprotein complexes has been technically challenging owing to the fragility and flexibility of these proteins. Many promising pre-clinical protein candidates have surfaced in recent years including native and/or stabilized soluble E2 protein (lacking transmembrane domain), multimeric assembly of soluble E2 using nano-particle display and soluble E1-E2 proteins complexed via chimeric dimerization motifs. Head-to-head comparison of these antigens is awaited to arrive at a definitive conclusion, but to date, the full-length E1-E2 protein complex remains the gold standard for subunit protein vaccines. Homogenous production, manufacturing, and scaling are anticipated to be logistical impediments in advancing this candidate, creating a gap for innovations that can overcome current limitations. mRNA delivery platform has been a game-changer in the vaccine space post-COVID. The present disclosure describes novel nucleic acid constructs, which encode antigens designed to leverage modern vaccine technology for an effective and commercially feasible HCV vaccine.

Determination of protein production and cell expression is utilized in many aspects and areas of study. Evaluation of the secretion of protein products or the expression of protein products on a cell surface is important information in many biochemical studies. Methods of evaluating and comparing protein production and protein expression are useful in such studies. The present disclosure provides for methods of evaluating the protein production of a transformed cell, and characterizing protein expression on the cell surface of a transformed cell, by differential manipulation of co-treated cell populations.

### BRIEF SUMMARY OF THE INVENTION

The present disclosure provides for a manmade nucleic acid construct that encompasses a nucleic acid sequence that encodes for a cell-surface expressible HCV E2 protein. It is further provided for constructs that enable the co-expression of HCV E1-E2 proteins in a cell. In particular, the constructs of the invention provide for the expression of E1-E2, as a polyprotein that is cleaved by signalase protein in the endoplasmic reticulum of a host cell to yield E1 protein and surface expressible E2 protein.

The present disclosure provides for nucleic acid constructs which encode for E1 protein and cell-surface expressible HCV E2 protein incorporated into a transformation vector construct.

The present disclosure provides for nucleic acid constructs which encode for E1 protein and cell-surface expressible HCV E2 protein incorporated into an RNA vaccine construct.

The present disclosure provides for nucleic acid constructs which encode for E1 protein and cell-surface expressible HCV E2 protein incorporated into a DNA vaccine construct.

The present disclosure provides for nucleic acid constructs which encode for E1 protein and cell-surface expressible HCV E2 protein incorporated into a hybrid DNA/RNA vaccine construct.

The present disclosure provides for nucleic acids which encode for E1 protein and cell-surface expressible HCV E2 protein incorporated into a protein expression construct.

The present disclosure provides for a nucleic acid construct encoding for E1 protein and cell surface expressible E2 protein, the nucleic acid comprising a nucleic acid sequence which encodes for an HCV E2 protein comprising a mutation which will result in the translated protein having the dual mutations of D728A and R730A (aaH77polyP reference numbering; FIG. 1).

The present disclosure provides for nucleic acid constructs which encode for E1protein and cell-surface expressible HCV E2 protein having a nucleic acid sequence which encode for an amino acid sequence which has the mutations of D728X+R730X′, where X and X′ can be either non-polar and/or aromatic amino acid residues (aaH77polyP reference numbering; FIG. 1, SEQ ID NO. 1, SEQ ID NO. 2).

The present disclosure provides for a nucleic acid construct encoding for E1 protein and cell surface expressible E2 protein, the nucleic acid comprising a nucleic acid sequence which encodes for an HCV E2 protein comprising a mutation which will result in the translated protein having the dual mutation from wild type of D728 and R730 (D728X+R730X′), wherein the mutant amino acid residue is selected from the non-polar amino acid group consisting of glycine, alanine, proline, cysteine, valine, leucine, isoleucine, methionine, tryptophan and phenylalanine (aaH77polyP reference numbering; FIG. 1, SEQ ID NO. 1, SEQ ID NO. 2).

The present disclosure provides for a nucleic acid construct encoding for E1protein and cell surface expressible E2 protein, the nucleic acid comprising a nucleic acid sequence which encodes for an HCV E2 protein comprising a mutation which will result in the translated protein having the dual mutation from wild type of D728 and R730 (D728X+R730X′), wherein the mutant amino acid residue is selected from the aromatic amino acid group consisting of tryptophan, phenylalanine and tyrosine (aaH77polyP reference numbering; FIG. 1, SEQ ID NO. 1, SEQ ID NO. 2).

The present disclosure provides for a nucleic acid construct encoding for E1 protein and cell surface expressible E2 protein, the nucleic acid comprising a nucleic acid sequence which encodes for an HCV E2 protein comprising a mutation which will result in the translated protein having the dual mutation from wild type of D728 and R730, where the nucleic acid is RNA. It is further provided for where the nucleic acid is DNA. It is also further provided for where the nucleic acid is a combination of RNA, DNA or artificial nucleotide analog (aaH77polyP reference numbering; FIG. 1, SEQ ID NO. 1, SEQ ID NO. 2).

The present disclosure provides for cell surface expressible E2 protein, the HCV E2 protein comprising a mutation from wild type of D728 and R730 (aaH77polyP reference numbering; FIG. 1, SEQ ID NO. 1, SEQ ID NO. 2).

The present disclosure provides for a vaccine which comprises a nucleic acid construct as described above.

The present disclosure provides for a vaccine for vaccinating a mammal against HCV, in need thereof, comprising at least one nucleic acid construct as described above, and a pharmaceutically acceptable carrier, excipient or diluent.

The present disclosure provides for a method of vaccinating a mammal against HCV, in need thereof, comprising administering a vaccine which comprises a nucleic acid construct encoding for E1 protein and cell surface expressible E2 protein, the nucleic acid comprising a nucleic acid sequence which encodes for an HCV E2 protein comprising a mutation which will result in the translated protein having the dual mutations of D728A and R730A and a pharmaceutically acceptable carrier, excipient or diluent (aaH77polyP reference numbering; FIG. 1, SEQ ID NO. 1, SEQ ID NO. 2).

The present disclosure provides for a method of vaccinating a mammal against HCV, in need thereof, comprising administering a vaccine which comprises a nucleic acid construct as described above and a pharmaceutically acceptable carrier, excipient or diluent, where the nucleic acid is RNA, DNA or a hybrid of RNA and DNA, or comprises synthetic nucleic acid analogs.

The present disclosure provides for a method of vaccinating a mammal against HCV, in need thereof, comprising administering a vaccine which comprises a HCV E2 protein as described above and a pharmaceutically acceptable carrier, excipient or diluent.

The present disclosure provides for methods for evaluating the protein expression of a protein produced by a cell comprising comparative assay of cell populations comprising a first test cell population in which cells are maintained under conditions to keep the cells intact; and a second test cell population maintained under conditions to make the cell membrane more permeable, such that when a protein detector means is administered; the first cell population allows for mostly cell surface protein detection; and in the second cell population detection of both extracellular and intracellular protein is enabled; such that comparison of the protein detector signal will yield evaluative results.

Thus the present disclosure provides for methods for comparative evaluation of protein expression in a cell, comprising:

Create at least two test cell samples by seeding cells in a suitable container with suitable media; Transfecting the cells with expression vector and incubating cells for sufficient time to allow for transformation and expression of protein from said expression vector; Fix the cells of the test cell samples; at least two sets of test cell samples are then subjected to divergent treatment at this point; Where a first test cell sample the cells are treated for detection of protein expression without permeabilization of the cells; Wherein a second test cell sample the cells are treated for detection of protein expression with permeabilization of the cells;

The test cell samples are then assayed for protein expression by detecting a signal which corresponds to the amount of protein detected; Wherein the signal detected in the first test cell sample will correspond to primarily cell surface expressed protein; And the signal detected in the second test cell sample will correspond primarily to total protein expression; Evaluating the results for comparison of signal detected.

The present disclosure provides for a method of coupling measurement of antigen exposure on the cell surface with antigenicity analysis using a broad panel of monoclonal antibodies against single, overlapping or independent epitopes of an antigenic protein or structure.

The present disclosure provides for the method of comparative protein evaluation as described above where the cells are selected from the group consisting artificial cell constructs, cell organoids, prokaryotic cells or eukaryotic cells. Where the suitable container is selected from the group consisting of liquid culture systems, solid culture systems, sample plates with multiple wells, individual tubes, culture vessels, culture bags, and living animals or organisms. Where the expression vector is suitable for use with the selected cells, selected from the group consisting of plasmid, viral, DNA, RNA or other suitable cell transforming vector which enables the test cell to express a protein encoded for by the expression vector. Where permeabilization of the cells is accomplished by a method selected from the group consisting of, chemical, biochemical, temperature, pressure, physical, agitation, or time. Where the detection of protein expression is done by a detection method selected from the group consisting of, antibody, antibody fragments, binding protein, chemical label, radiometric, fluorescence measurement, flow cytometry, photo metric, color metric, mass spectroscopy, magnetic resonance and sample mass measurement. Where the detection is end-point or kinetic, with the latter readout employed to circumvent issues related to assay saturation. Wherein the process is partially or fully automated.

Thus, in a preferred embodiment the present disclosure provides for one method of assay as depicted in FIG. 2.

As described, the present disclosure provides for a method which encompasses:
a. Create at least two, (or two or more), substantially identical test cell samples by seeding cells in a suitable container with suitable media;
b. Transfecting the cells with expression vector and incubating cells for sufficient time to allow for transformation and expression of protein from said expression vector;
c. Fix the cells of the test cell samples;
d. Subject the substantially identical test cell samples to divergent treatment;
e. Where a first test cell sample (or group of test samples) the cells are treated for detection of protein expression without permeabilization of the cells;
f. Wherein a second test cell sample (or group of test samples) the cells are treated for detection of protein expression with permeabilization of the cells;
g. The test cell samples are then assayed for protein expression by detecting a signal which corresponds to the amount of protein detected;
h. Wherein the signal detected in the first test cell sample will correspond to primarily cell surface expressed protein;
i. And the signal detected in the second test cell sample will correspond primarily to total protein expression;
j. Evaluating the results for comparison of signal detected.

Therefore, the present disclosure provides for a method which comprises the steps of:
a. Create two substantially identical test cell samples by seeding cells in a suitable container with suitable media;
b. Transfecting the cells of the test cell samples with expression vector and incubating the cells for sufficient time to allow for transformation and expression of protein from said expression vector;
c. Fix the cells of the test cell samples;
d. Subject the substantially identical test cell samples to divergent treatment;
e. Where a first test cell sample the cells are treated for detection of protein expression without permeabilization of the cells;
f. Wherein a second test cell sample the cells are treated for detection of protein expression with permeabilization of the cells;
g. The test cell samples are then assayed for protein expression by detecting a signal which corresponds to the amount of protein detected;
h. Wherein the signal detected in the first test cell sample will correspond to primarily cell surface expressed protein;
i. And the signal detected in the second test cell sample will correspond primarily to total protein expression;
j. Evaluating the results for comparison of signal detected.

It is understood that the described method can be utilized to compare more than two test cell samples, depending upon the number of groups created and subjected to divergent modes of treatment as to cell membrane permeability.

### BRIEF DESCRIPTION OF THE DRAWINGS

A better understanding of the present invention will be obtained upon reference to the following description in conjunction with the accompanying drawings.

FIG. 1 Describes and depicts the protein sequence of HCV E1E2 construct with highlight of residues of interest. E1 and E2 protein amino acid numbering in first column, E1and E2 amino acid numbering as in polypeptide in second column (aaH77polyP) SEQ ID NO. 1 corresponds to HCV_1a_H77_E1, SEQ ID NO. 2 corresponds to HCV 1a H77 E2.

FIG. 2 Describes assay design for screening mutants.

FIG. 3 Depicts data which compares data showing E2 levels on the cell surface vs. total cellular signal, across different E1E2 constructs as tested with E2 reactive antibodies that target linear epitopes. FIG. 3A antibody HC33.1 cell surface vs. (3B) total cellular signal; FIG. 3C antibody HC33.4 (against E2-domain E) cell surface vs. (3D) total cellular signal.

FIG. 4 Depicts data showing cell surface vs. total cellular signal for E1 protein, E2 protein and E1-E2 heterodimer for different constructs as measured by IGH526, AR3A and AR4A antibodies, respectively. (antibodies provided by Mansun Law, The Scripps Research University, USA.) FIG. 4A depicts data for E1 measured by IGH526 cell surface vs. (4B) total cellular signal; FIG. 4C depicts data for E2 Domain B measured by AR3A cell surface vs. (4D) total cellular signal; FIG. 4E depicts data for E1-E2 measured by AR4A cell surface vs. (4F) total cellular signal.

FIG. 5 Antibodies targeting E2 antigenic Domain A. Depicts data which compares reactivity of D728A+R730A mutations with wild type to a broad antibody panel targeting different antigenic regions of E2 protein (aaH77polyP reference numbering; FIG. 1). FIG. 5A-D shows data for antibodies targeting E2 antigenic domain A; FIG. 5A cell surface vs. (5B) total cellular signal (CBH4D); FIG. 5C cell surface vs. (5D) total cellular signal (CBH4G).

FIG. 6 Antibodies targeting E2 antigenic Domain B (AR3). Depicts data which compares reactivity of D728A+R730A mutations with wild type to a broad antibody panel targeting different antigenic regions of E2 protein (aaH77polyP reference numbering; FIG. 1). FIG. 6A-D shows data for broadly neutralizing antibodies (bNAbs) targeting E2 antigenic domain B; FIG. 6A cell surface vs. (6B) total cellular signal (CBH2); FIG. 6C cell surface vs. (6D) total cellular signal (CBH5).

FIG. 7 Antibodies targeting E2 antigenic Domain C. Depicts data which compares reactivity of D728A+R730A mutations with wild type to a broad antibody panel targeting different antigenic regions of E2 protein (aaH77polyP reference numbering; FIG. 1). FIG. 7A-D shows data for antibodies targeting E2 antigenic domain C; FIG. 7A cell surface vs. (7B) total cellular signal (CBH7); FIG. 7C cell surface vs. (7D) total cellular signal (CBH23).

FIG. 8 Antibodies targeting E2 antigenic Domain D. Depicts data which compares reactivity of D728A+R730A mutations with wild type to a broad antibody panel targeting different antigenic regions of E2 protein (aaH77polyP reference numbering; FIG. 1). FIG. 8A-D shows data for bNAbs targeting E2 antigenic domain D; FIG. 8A cell surface vs. (8B) total cellular signal (HC84.26AM); FIG. 8C cell surface vs. (8D) total cellular signal (HC84.22).

FIG. 9 Antibodies targeting E2 antigenic Domain E. FIG. Depicts data which compares reactivity of D728A+R730A mutations with wild type to a broad antibody panel targeting different antigenic regions of E2 protein (aaH77polyP reference numbering; FIG. 1). FIG. 9A-B shows data for bNAbs targeting E2 antigenic domain E; FIG. 9A cell surface vs. (9B) total cellular signal (HC33.4).

FIG. 10 Antibodies targeting E2 antigenic region stabilized by E1 (E1-E2). Depicts data which compares reactivity of D728A+R730A mutations with wild type to a broad antibody panel targeting different antigenic regions of E2 protein (aaH77polyP reference numbering; FIG. 1). FIG. 5F shows data for bNAbs targeting E2 antigenic region stabilized by E1; FIG. 10A cell surface vs. (10B) total cellular signal (H202); FIG. 10C cell surface vs. (10D) total cellular signal (H205).

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As used herein, the term nucleic acid construct refers broadly to artificially constructed nucleic acid sequences which comprise natural nucleic acid residues both DNA and RNA, or which comprise a mixture of the two. It is further considered that artificial nucleotides may be used as substitutes for natural nucleic acid residues as well.

As used herein, the term “encodes for” is meant to refer broadly to the conversion of a given nucleic acid construct into the corresponding amino acid sequence as provided for by all understood operating coding triplets, both DNA and RNA or a mix thereof.

Thus where an amino acid sequence is provided, it is understood that the nucleic acid constructs which encode for such amino acid sequence can encompass variations and substitutions in coding by specific nucleic acid residues, such that the triplet codon is translated into the corresponding amino acid as specified in the recited amino acid sequence.

The phrase “pharmaceutically acceptable carrier, excipient or diluent” references broadly the know art of vaccine production and delivery. It is contemplated that delivery of such vaccines may include various packaging forms and various routes of administration, for example i.m., i.p., oral, surface, i.v. etc. as are known in the art. The requirements for carriers, excipients and diluents will vary somewhat depending upon the route of administration. In the case of nucleic acid vaccines such as mRNA, DNA or the like, the packaging may require use of other forms of micro-encapsulation which is contemplated under the use of the phrase “pharmaceutically acceptable carrier, excipient or diluent.”

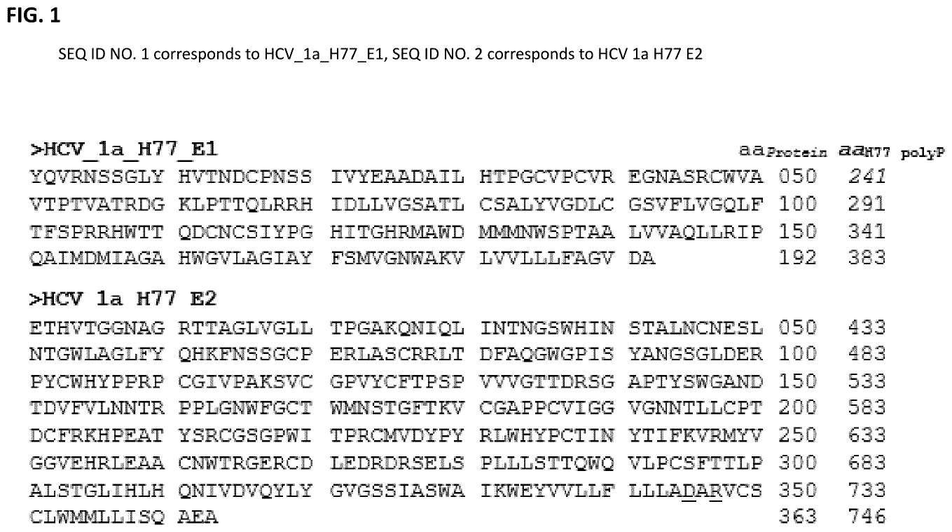

The amino acid numbering convention used herein is with reference to the viral polypeptide amino acid sequence, and E1, E2 protein amino acids are numbered accordingly (aaH77polyP reference numbering; FIG. 1, SEQ ID No. 1 and SEQ ID NO. 2).

As described, the present disclosure provides for the identification of specific mutation targets in the E1, E2 protein amino acid sequence. Functional variants may be constructed utilizing methods know in the art. The term “functional variant” refers to a nucleotide, peptide, a polypeptide, or a protein having substantial or significant sequence identity or similarity to the reference nucleotide, peptide or polypeptide, which functional variant retains the biological activity of the reference sequence of which it is a variant. Functional variants encompass, for example, those variants of a sequence (the parent sequence) that retain the ability to exhibit the properties (such as, for example, binding functionality) and to a similar extent, the same extent, or to a higher extent, as the parent sequence.

In reference to a nucleic acid sequence encoding the peptide or polypeptide, in some embodiments a nucleic acid sequence encoding a functional variant of the peptide or is about 10% identical, about 25% identical, about 30% identical, about 50% identical, about 65% identical, about 75% identical, about 80% identical, about 90% identical, about 95% identical, or about 99% identical to the nucleic acid sequence encoding the parent sequence.

A method for the comparative evaluation of protein expression by a cell is provided for by this disclosure. The detection of protein production by a cell encompasses the use of many readily known methods and detection systems utilizing signal linked antibody, antibody fragment, antibody binding domain constructs and the like. Detection systems include signal linked receptor ligands, proteins, nucleic acids or other such bound signals for detection. Means for production of detectable signal are know, and include and are not limited to enzyme color activation, radio isotopes, metal atoms, and other such signal methods. Detection of such signal can be accomplished by know methods which correspond to the signal system utilized, such as and not limited to color detection, fluorescence detection, radioactive emission detection, and even cell sorting and other such flow cytometric systems. In addition, it is known that secondary and tertiary binding chains can be utilized to detect and/or amplify detection where a first ligand which binds to a desired protein to be measured is then subsequently bound by a second binding protein or signal linked detection system. The described method can be used to measure antigen exposure on cell surface along with analysis of the antigenic landscape, and therefore can be used as a quantitative and rapid screening tool for antigen discovery and vaccine design in a format amenable to multiplexing and automation.

Cell culture methods suitable for the practice of the disclosed method encompass all methods which will result in an isolated measurable population of target cells. The target cells can remain in solution, or affixed to a surface. Manipulation and treatment of the target cells can be accomplished in either solution or affixed to a surface or contained within a matrix of material such as an artificial organoid construct and/or biogel matrix.

Methods for transfecting and expression of protein in a target cell is known in the art and are suitable for use in the method described. It is envisioned that cells can be transformed in vitro, and in vivo via the appropriate vector or transformation system.

The overall method and operative steps are suitable for incorporation into a semi-automated or fully automated system where manipulation of samples and signal detection can be conducted by mechanical processes.

The field of mRNA vaccinology offers a tremendous opportunity to produce HCV vaccine antigens “on demand, on site”, within the vaccinees, in an unperturbed native state. This helps circumvent the arduous process of large-scale production of the delicate HCV E1-E2 surface transmembrane proteins in their subsequent purification to obtain a homogenous formulation. However, the membrane bound E1-E2 proteins are primarily retained inside the cells upon expression, due to the presence of a static endoplasmic reticulum (ER) retention signal. This is in stark contrast to SARS-COV-2 spike or influenza virus hemagglutinin proteins that naturally traffic to the plasma membrane, have direct interface with the immune system and are paragons for mRNA delivered vaccines. We envisioned that the poor visibility of the HCV antigens to the immune system, could potentially impair their appropriate engagement with the immune cells and have direct consequences for vaccine efficacy and dosage. The exposure to the immune system of the mRNA expressed, HCV E1/E2 proteins can potentially be enhanced either by using soluble/secreted antigen(s) or re-routing these protein(s) to enable their display on the cell surface. We developed the latter approach with the idea that the cell surface targeted full-length proteins with transmembrane domains (TMDs) are likely to mimic native antigen more closely in 3D structure and top- down polarity with the possibility that the plasma membrane displayed antigens are more uniform and homogenous compared to mRNA expressed or purified soluble antigen counterparts.

This disclosure describes the a) approach to re-route the major HCV antigenic glycoprotein (E2) to the plasma membrane b) assay to concurrently screen the cell surface bound vs total antigen expression, antigenic landscape, and oligomeric status of the HCV proteins c) assessment of the antigenic profile using a broad panel of HCV antibodies. The cell surface displayed antigen(s) can be further engineered to tune the “distracted” immune response in the direction of key broadly neutralizing epitopes. In summary we have harnessed the power of a mRNA platform and optimized the display of HCV antigens for an efficacious vaccine.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

### Part 1: Rationale for E1E2 Co-Expressing Construct with Mutations in E2 Protein

Cell surface expression of HCV E2 protein is facilitated by the construction of an E1E2 polyprotein encoding genetic construct (FIG. 1). While the E1 and E2 proteins are cleaved in the ER by host cell signalase, it is believed that the presence of E1 facilitates the proper folding of E2 and vice versa.

It is believed that the wild type E1 and E2 proteins, which are primarily retained in the cell, may provide for basal level immune response through cell lysis, expression on extracellular vesicles as well as their minimal presentation on the cell surface (where ER retention is not absolute).

It was our belief and we have shown that the display of E2 protein on the cell surface (via the mutations described in this disclosure) in addition to intracellular pools of E1and mutant E2 protein is possible, and believe that it will provide for a more dynamic and robust pathway to stimulate the immune system. This presentation will yield better immunological results than the wild type presentation which is mainly via intracellular expression of E1 and E2 proteins.

### Part 2: Cell Surface Presentation of HCV E2 Antigens

Mutations of D728A or R730A redirect E2 from ER to the cell surface. In the present disclosure we demonstrate that the combined mutation encoding for both D728A and R730A (D728A+R730A) provides for efficient cell surface expression of the E2 protein (aaH77polyP reference numbering; FIG. 1).

FIG. 1 depicts the protein sequence of HCV E1E2 protein, expressed as a polyprotein. (E1 and E2 are expressed as a polyprotein, individual protein amino acid (aa) numbering on the left is labeled aaProtein and numbering of aa in the context of the full-length viral polyprotein is indicated on the right as aa H77 polyP). The polyprotein is cleaved into individual proteins by host cell signalase in the endoplasmic reticulum (ER) of recipient cells. E1 translocation into the ER is initiated by signal sequence (not shown). Signal sequence could either be native from HCV core protein

(MVGCSFSIFLLALLSCLTVPASA) or derived from tissue plasminogen protein (tPA) (MDAMKRGLCCVLLLCGAVFVDSVTG). The amino acid sequence depicted in FIG. 1 represents Genotype 1a, H77 strain with viral polyprotein aa numbering indicated on the far right (aaH77 polyP). Other genotypes and strains of HCV may be similarly mutated according to the teaching of the present disclosure. In FIG. 1 residues of interest are underlined (D728 and R730 in E2 protein) (aaH77polyP reference numbering; FIG. 1), SEQ ID NO. 1 corresponds to HCV_1a_H77_E1, SEQ ID NO. 2 corresponds to HCV 1a H77 E2.

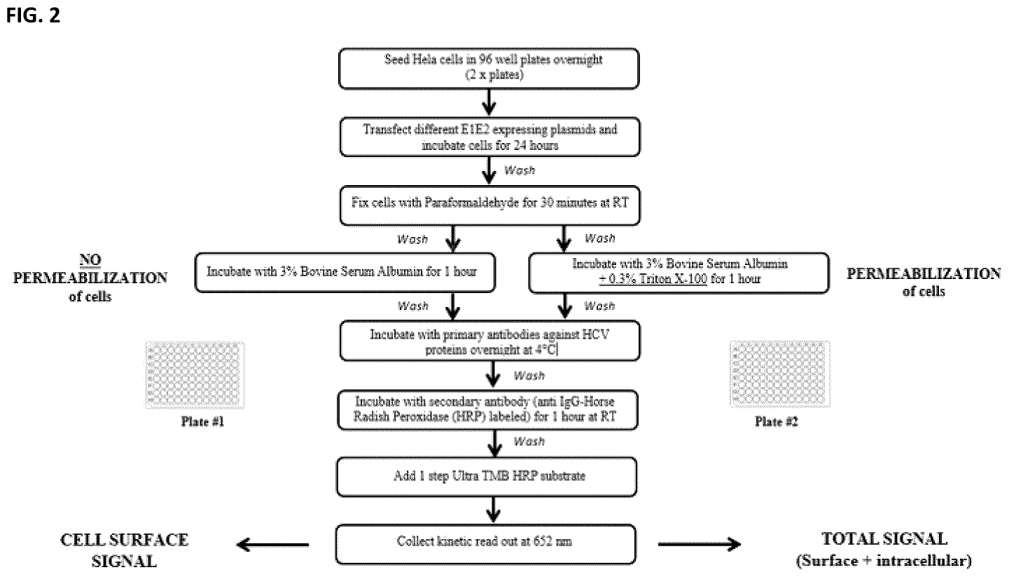

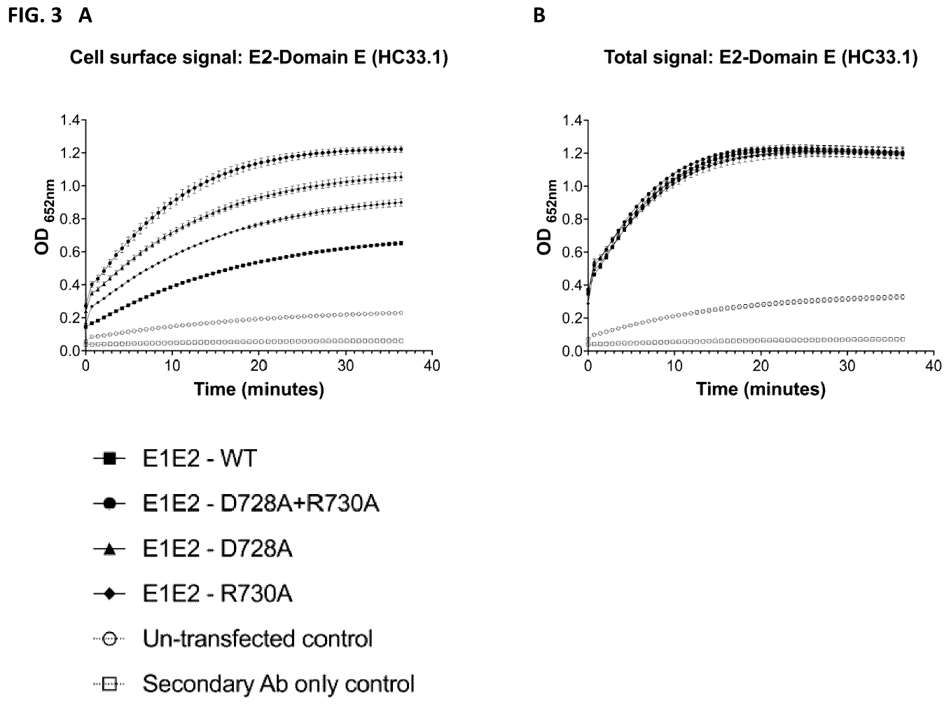

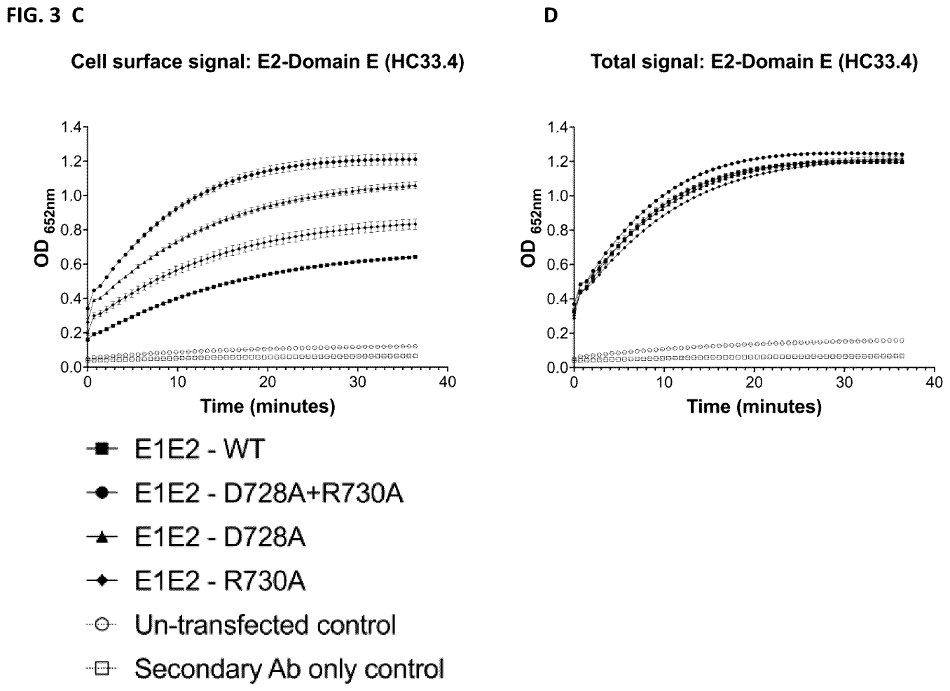

Using the assay design described in FIG. 2, we show that the D728A+R730A combined mutation leads to elevated levels of E2 on the cell surface when compared to the single mutations alone, or the wild type (FIGS. 3-5). The assay is a newly designed in-cell ELISA for HCV antigens (aaH77polyP reference numbering; FIG. 1).

FIG. 3 shows data which compares E2 levels on the cell surface across different constructs. Cell surface vs. total signal is measured with E2-domain E antibodies, HC33.1 and HC33.4. These antibodies each target a linear epitope and therefore can be used to account for expression levels of E2 protein in the cell. Total signal for different constructs is similar, suggesting that E2 is expressed in equal amounts in the cell and demonstrate that these mutations do not have any deleterious impact on protein expression. The cell surface signal follows the order D728A+R730A>D728A>R730A>Wild type E1E2 (aaH77polyP reference numbering; FIG. 1).

It is envisioned that the targeted modification of protein residue D728 and R730 can be conducted substituting the wild-type Aspartic Acid (D) and Arginine (R) with other amino acid residues or amino acid analogs which enhance cell surface expression of E2 (aaH77polyP reference numbering; FIG. 1).

Other neutral amino acids and/or aromatic amino acids, such as glycine, proline, cysteine, valine, leucine, isoleucine, methionine, tryptophan, phenylalanine, and tyrosine may be as effective as the demonstrated alanine mutation.

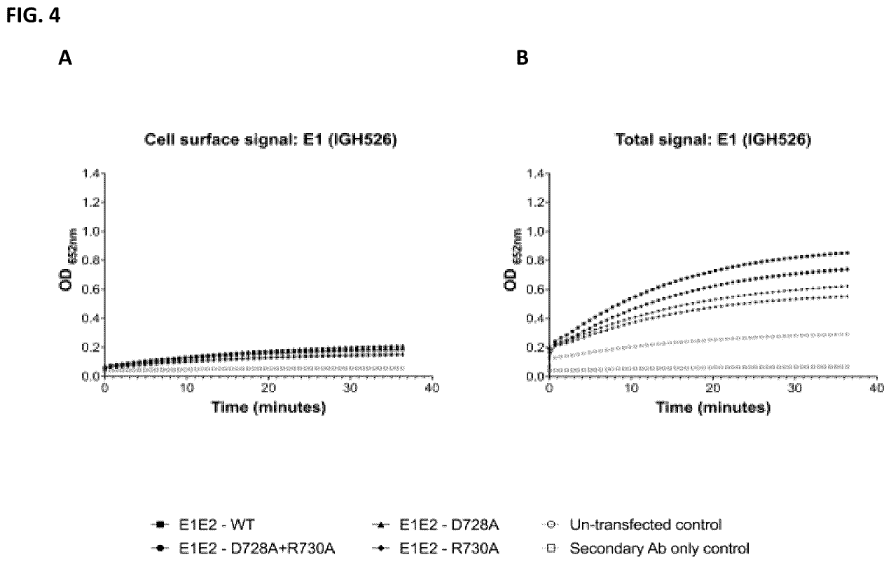

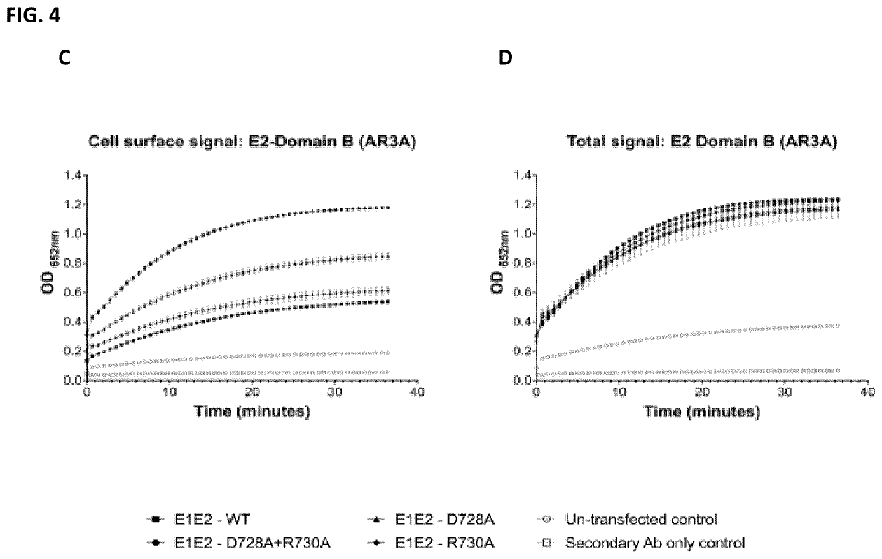

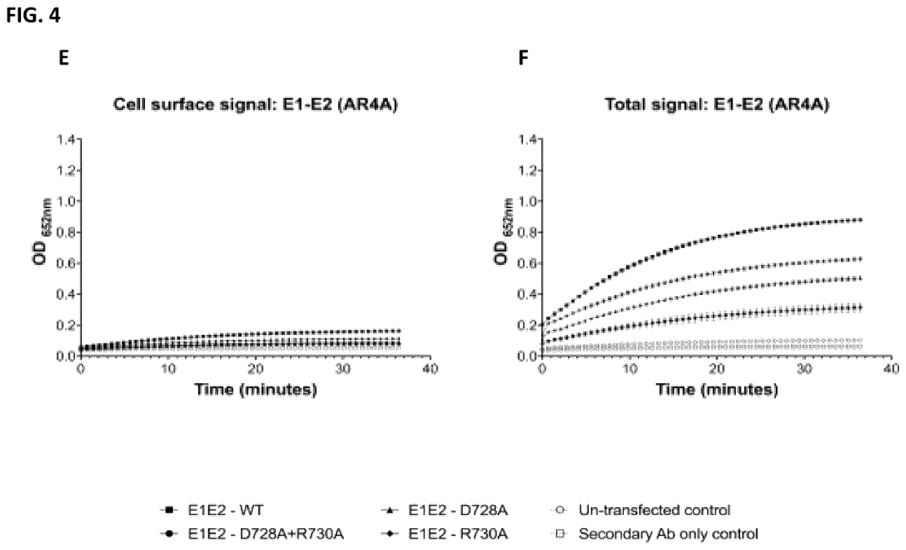

FIG. 4 compares data for cell surface vs. total signal for E1 protein, E2 protein, and noncovalent E1-E2 heterodimeric protein complex across different constructs as measured by antibodies AR3A (against E2-domain B), AR4A (against E2 epitope stabilized by E1) and IGH526 (against E1). The data shows that while E2 protein is robustly expressed on the surface in the case of mutants (D728A+R730A>D728A>R730A>Wild type), the E1 protein is present at exceedingly low levels in case of all constructs including the mutants and wild type. Furthermore, the mutations reduce AR4A signal both inside the cell and on the cell surface suggesting their negative impact on the heterodimerization of E1 and E2. The AR4A signal, which is reflective of E1-E2 heterodimers, follows the order Wild type E1E2>R730A>D728A>D728A+R730A (aaH77polyP reference numbering; FIG. 1).

Mutations in the charged residues of E2 transmembrane domain (D728A+R730A) therefore only target E2 to the cell surface. E1 is still retained in the ER and the heterodimerization of E1-E2 is disrupted either as a consequence of differential localization of these proteins or as a direct consequence of the mutations.

Despite the inability of E1 to traffic to the cell surface, expressing E2 with D728A+R730A mutations in the natural context of a polyprotein containing E1, has potential advantages such as E1 ensuring the proper folding of E2 and basal immune response against intracellular E1 (aaH77polyP reference numbering; FIG. 1). Given that the E2 protein is the primary driver of antibody response, its presence on the cell surface is a must, with intracellular E1 being an added bonus.

Ongoing experiments are gauging the effect of substitution of D728 residue to aromatic amino acid (tryptophan, phenylalanine, and tyrosine) which we hypothesize to not-only increase E2 expression on the cell but also preserve E1-E2 heterodimerization. D728 residue in the E2 TMD likely forms an ionic interaction with K370 in the E1 TMD which is possibly abrogated when D728 is mutated to alanine. Our experimental goal is to leverage cation-x interactions by substituting D728 to tryptophan, phenylalanine, and tyrosine. These efforts could yield an E1E2 candidate with surface expressible E2 protein with intact AR4A reactivity, indicative of preserved E1E2 interaction (aaH77polyP reference numbering; FIG. 1).

### Part 3: Cell Surface Expressible E2 Maintains a Near Native Antigenic Landscape

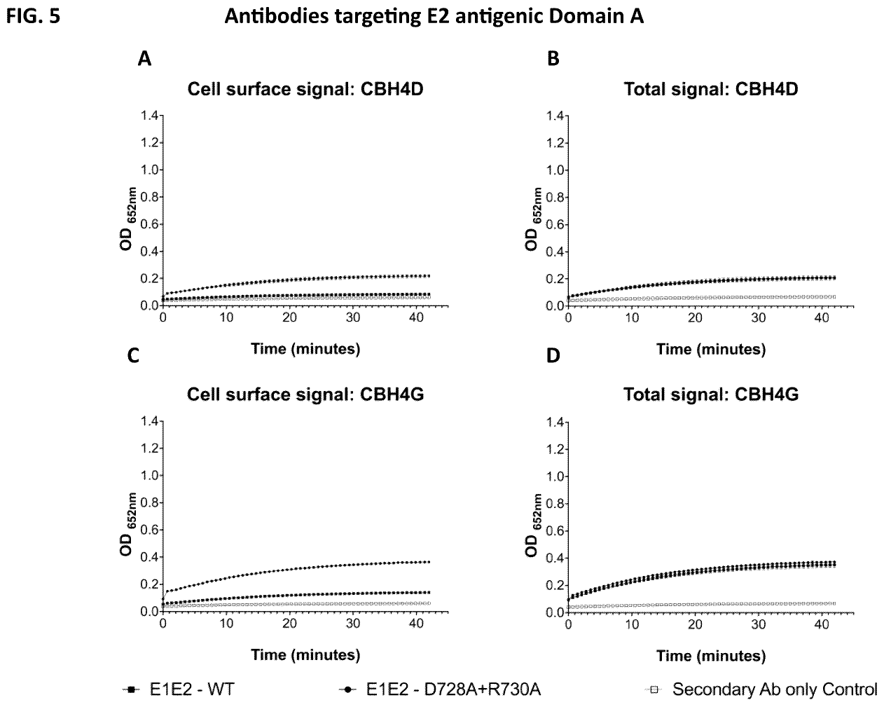

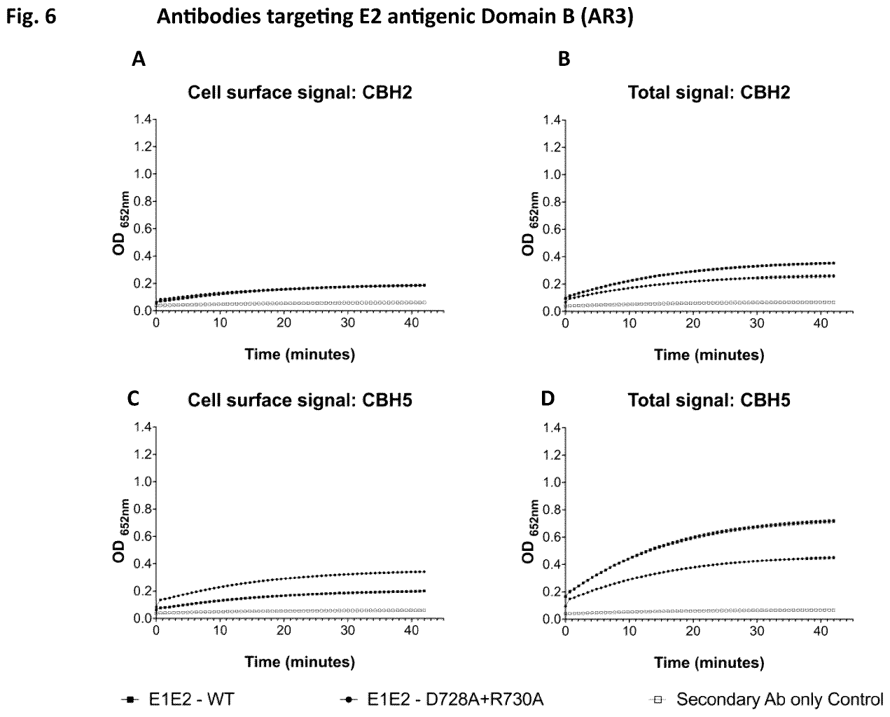

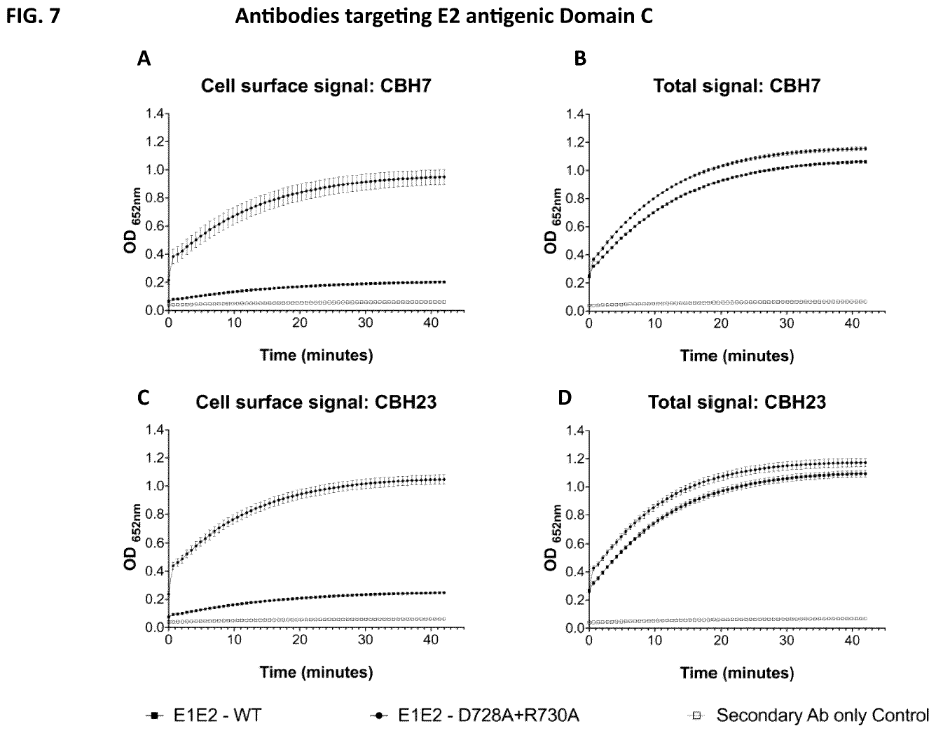

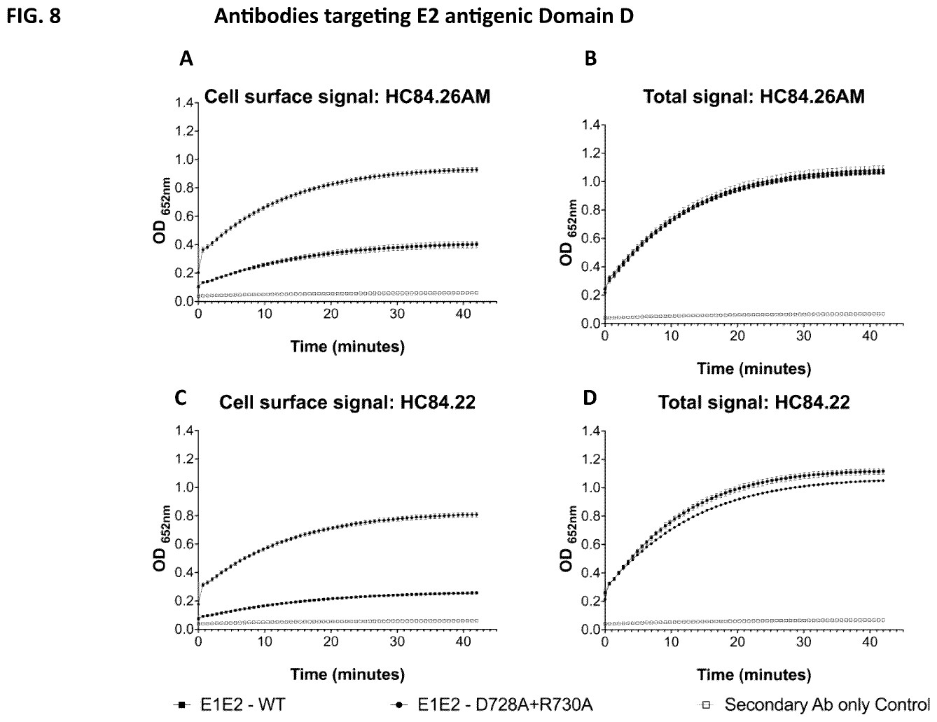

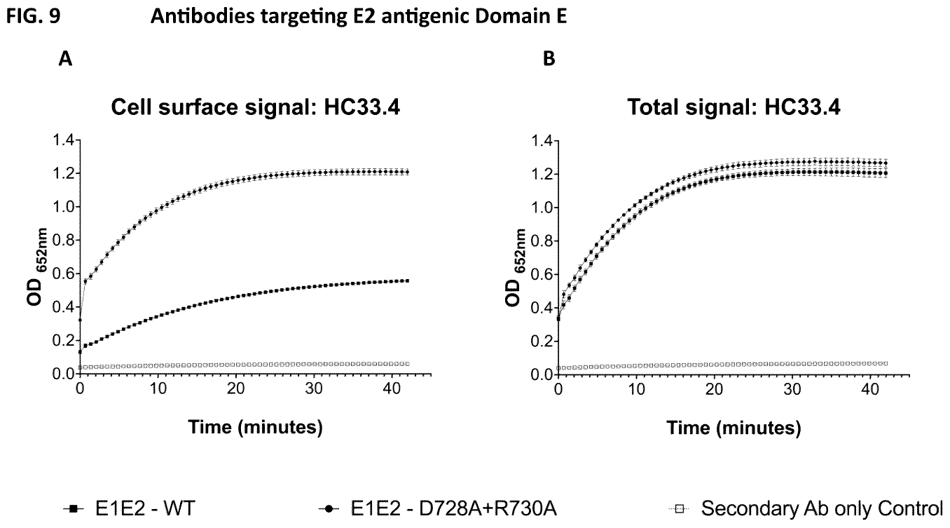

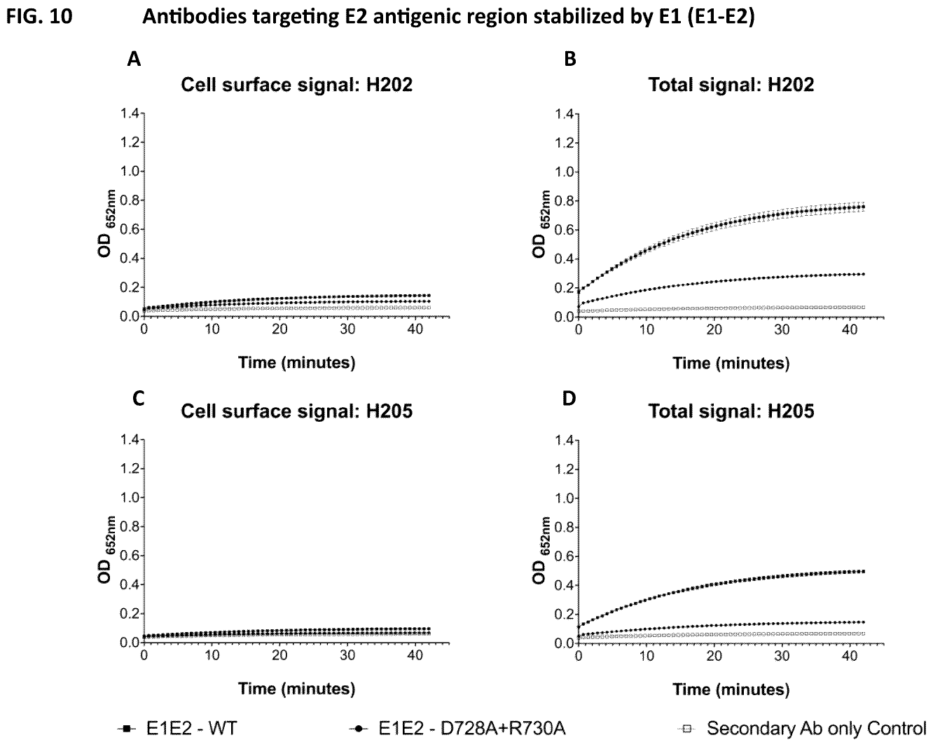

The present disclosure demonstrates that the combined D728A+R730A mutations provides for efficient cell surface expression of the E2 protein without deformation or disruption of majority of the antigenic epitopes on the protein. As shown in FIG. 5(A-E), a panel of antibodies targeting a spectrum of E2 antigen sites, still bind to the D728A+R730A surface expressed E2 protein (aaH77polyP reference numbering; FIG. 1). These includes important bNAbs targeting critical epitopes in E2 Domain B, Domain D, and Domain E, with the latter two being completely unperturbed and the former showing only modest changes (reflected in total signal) (FIG. 5B, 5D, 5E). As described above, the exception is AR4A/H202/H205 class of antibodies that recognize E2 epitopes stabilized by E1 protein (E1-E2 epitopes), which show greatly reduced binding to the mutant E2 protein (FIG. 5F).

Furthermore, D728+R730A mutations increase cell surface signal for E2, as measured by all E2 specific antibodies (aaH77polyP reference numbering; FIG. 1). Thus, as demonstrated by diverse antibodies binding to the cell-surface expressed E2 protein, the cell expressed E2 protein retains most recognizable epitope conformations.

Together the data from a broad group of antibodies demonstrates enhanced surface expression and intact reactivity of the engineered E2 antigen (D728A+R730A) qualifying this construct as a preclinical vaccine candidate and setting the stage for animal trials (aaH77polyP reference numbering; FIG. 1).

Accordingly, immunogenicity assessment in animals are in progress to assess the antibody response against mRNA encoded surface expressed E1E2 (D728A+R730A) versus mRNA encoded wild type E1E2 constructs (aaH77polyP reference numbering; FIG. 1).

### Materials and Methods

Examples of the general state of knowledge in the art can be found, for example, in Essential Cell Biology by Alberts et al (2023); Molecular Biology by Zlatanova et al., (2023); Molecular Biotechnolgy-Principles and Applications of Recombinant DNA by Glick and Patten (2022); Molecular Biology of the Cell by Alberts et al., (7^{th} Ed. 2022), and Molecular Biology of the Gene by Watson et al., (7^{th} Ed. 2013).

Materials and methods for making a desired nucleic acid sequence are well known in the art. Similarly, materials and methods for the translation and expression of a nucleic acid sequence into protein fragments all the way up to complete functional proteins are known in the art. Materials and methods for making a nucleic acid sequence which encodes for a cell-surface expressible glycoprotein are known in the art.

Materials and methods for making constructs for the transmission of desired nucleic acid sequences which encode for particular signals or proteins, and the ability for such transformations to be incorporated into a targeted cell are known in the art. While outcome or such methods are subject to unpredictable effects, standard transformation vector constructs for many types of target cells are known in the art. For example, the construction of RNA vaccines (Schlake et al., 2012, RNA Biol. 9(11): 1319-1330), construction of DNA vaccines (Lee et al., 2018, Acta Biomater 80:31-47), construction of hybrid DNA/RNA vectors or other such engineered tools (Nora et al., 2019, Microb Biotechnol 12(1): 125-147), and/or the construction of novel non-viral protein expression vectors (Sainz-Ramoz et al., 2021 Int J Mol Sci 22(14): 7545) are known in the art.

### General

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

In the above description, numerous specific details are set forth to provide a thorough understanding of the present disclosure. Particular examples may be implemented without some or all of these specific details and it is to be understood that, unless otherwise stated, aspects hereof are not limited to particular biological systems or particular species of bacteria or plants, which can, of course, vary but remain applicable in view of the data provided herein.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It should be emphasized that the following disclosures are put forth so as to provide those of ordinary skill in the art with a complete disclosure and description of how the composition of matter, e.g., the mutant strains in this disclosure, and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

The terms “about,” “approximate,” “at or about,” and “substantially,” when referring to a number or a numerical value or range (including, for example, whole numbers, fractions, and percentages), means that the number or numerical range referred to is an approximation within experimental variability (or within statistical experimental error). That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated +1%-15% variation of the stated number or numerical range (e.g., +/−5% to 15% of the recited value), provided that one of ordinary skill in the art would consider equivalent to the recited value (e.g., having the same function or result) and unless otherwise indicated or inferred. Similarly, when values are expressed as approximations, by use of the antecedent “about,” it will be understood that the particular value forms a further aspect. For example, if the value “about 10” is disclosed, then “10” is also disclosed.

When ratios, ranges, concentrations, amounts, and other numerical data are expressed herein in a range format, all combinations and sub-combinations of such ranges and specific embodiments therein are intended to be included. It will be further understood that the endpoints of each of the ranges are significant both in relation to the other endpoint, and independently of the other endpoint.

Additionally, it is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and
scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

Those skilled in the art will recognize that numerous modifications can be made to the specific implementations described above. The implementations should not be limited to the particular limitations described. Other implementations may be possible.

It should be understood by those skilled in the art that various alternatives to the embodiments described herein may be employed in practicing the claims without departing from the spirit and scope as defined in the following claims.

What is claimed is:

1. A nucleic acid construct encoding for El protein and cell surface expressible E2 protein, comprising a nucleic acid sequence which encodes for an HCV E2 protein comprising a mutation which will result in the translated protein having the dual mutations of D728X and D730X′ in the amino acid sequence of SEQ ID NO. 1 and SEQ ID NO 2 (aaH77polyP reference numbering; FIG. 1), where X and X′ are selected from the group consisting of non-polar or aromatic amino acid residues.

2. The nucleic acid construct of claim 1 where in X and X′ are selected from the group of non-polar amino acids consisting of glycine, alanine, proline, cysteine, valine, leucine, isoleucine, methionine, tryptophan and phenylalanine.

3. The nucleic acid construct of claim 1 wherein X and X′ are selected from the group of aromatic amino acids consisting of tryptophan, phenylalanine and tyrosine.

4. The nucleic acid construct of claim 1 encoding for an HCV E2 protein comprising a mutation that will result in the translated protein having the dual mutations of D728A and D730A in the amino acid sequence of SEQ ID NO. 2.

5. A cell surface expressible HCV E2 protein, comprising a mutation that will result in the protein having the dual mutations of D728X and D730X′ in the amino acid sequence of FIG. 1 SEQ ID NO. 2, where X and X′ are selected from the group consisting of non-polar or aromatic amino acid residues.

6. The cell surface expressible HCV E2 protein of claim 5 wherein X and X′ are selected from the group of non-polar amino acids consisting of glycine, alanine, proline, cysteine, valine, leucine, isoleucine, methionine, tryptophan and phenylalanine.

7. The cell surface expressible HCV E2 protein of claim 5 wherein X and X′ are selected from the group of aromatic amino acids consisting of tryptophan, phenylalanine and tyrosine.

8. A method for evaluating the protein expression of a protein produced by a cell comprising a comparative assay of cell populations comprising a first test cell population in which cells are maintained under conditions to keep the cells intact; and a second test cell population maintained under conditions to make the cell membrane more permeable, such that when a protein detector means is administered; the first cell population allows for mostly cell surface protein detection; and in the second cell population detection of both extracellular and intracellular protein is enabled; such that comparison of the protein detector signal will yield evaluative results.

9. A method of claim 8 for comparative evaluation of protein expression in a cell, comprising:
a. Create two substantially identical test cell samples by seeding cells in a suitable container with suitable media;
b. Transfecting the cells with expression vector and incubating cells for sufficient time to allow for transformation and expression of protein from said expression vector;
c. Fix the cells of the test cell samples;
d. Subject the substantially identical test cell samples to divergent treatment;
e. Where a first test cell sample the cells are treated for detection of protein expression without permeabilization of the cells;
f. Wherein a second test cell sample the cells are treated for detection of protein expression with permeabilization of the cells;
g. The test cell samples are then assayed for protein expression by detecting a signal that corresponds to the amount of protein detected;
h. Wherein the signal detected in the first test cell sample will correspond to primarily cell surface expressed protein;
i. And the signal detected in the second test cell sample will correspond primarily to total protein expression;
j. Evaluating the results for comparison of signal detected.

10. The method of claim 9 where the cells are selected from the group consisting artificial cell constructs, cell organoids, prokaryotic cells or eukaryotic cells.

11. The method of claim 9 where the suitable container is selected from the group consisting of liquid culture systems, solid culture systems, sample plates with multiple wells, individual tubes, culture vessels, culture bags, and living animals or organisms.

12. The method of claim 9 where the expression vector is suitable for use with the selected cells, selected from the group consisting of plasmid, viral, DNA, RNA or other suitable cell transforming vector which enables the test cell to express a protein encoded for by the expression vector.

13. The method of claim 9 where the permeabilization of the cells is accomplished by a method selected from the group consisting of, chemical, biochemical, temperature, pressure, physical, agitation, or time.

14. The method of claim 9 where the detection of protein expression is done by a detection method selected from the group consisting of, antibody, antibody fragments, binding protein, chemical label, radiometric, fluorescence measurement, flow cytometry, photometric, color metric, mass spectroscopy, magnetic resonance and sample mass measurement.

15. The method of claim 9 wherein the process is partially or fully automated.
